(19) United States
(12) Patent Application Publication
(10) Pub. No.: US 2011/0288649 A1
(43) Pub. Date: Nov. 24, 2011

(54) INTRODUCTION INSTRUMENT

(75) Inventors: Adam R. Ratzel, Warsaw, IN (US); Mark A. Wise, Warsaw, IN (US)

(73) Assignee: ZIMMER, INC., Warsaw, IN (US)

(21) Appl. No.: 12/783,971

(22) Filed: May 20, 2010

(51) Int. Cl. A61F 2/34

(52) U.S. Cl. 623/22.24

(57) ABSTRACT

An introduction instrument for inserting a liner into an acetabular shell is disclosed. The instrument includes a suction device which may be selectively released once the liner is appropriately situated in the acetabular shell. Such selective release prevents disruption of the positioning of the liner and/or the acetabular shell.

### BACKGROUND

1. Field of the Disclosure

This disclosure relates to an instrument used for orthopaedic surgery, and more particularly to an introduction instrument for inserting a liner into an acetabular shell.

2. Description of the Related Art

Acetabular cups may include a metallic acetabular shell and a liner. The liner may be formed of various materials such as metal, polyethylene, and ceramic. During an exemplary orthopaedic surgical procedure, the acetabular shell is implanted into an acetabulum of a patient. Once the shell is positioned, the liner is secured to the acetabular shell to provide an articulating surface for articulating with a natural femoral head or a femoral head prosthesis. Sufficient fixation and correct placement of the liner relative to the acetabular shell facilitates proper functioning and longevity of the acetabular cup.

### SUMMARY

The present disclosure provides an introduction instrument for inserting the liner into the acetabular shell. The instrument includes a suction device which may be selectively released once the liner is appropriately situated in the acetabular shell. Such selective release prevents disruption of the positioning of the liner and/or the acetabular shell.

### BRIEF DESCRIPTION OF THE DRAWINGS

The above-mentioned and other features of this disclosure will become more apparent and the disclosure itself will be better understood by reference to the following description of embodiments of the disclosure taken in conjunction with the accompanying drawings, wherein:

FIG. 1 is a perspective view of an introduction instrument according to the present disclosure;

FIG. 2 is a partial cross-sectional perspective view of the introduction instrument of FIG. 1;

FIG. 3 is a fragmentary cross-sectional view of a portion of the introduction instrument of FIG. 2;

FIG. 4 is a perspective view of a suction cup of the introduction instrument of FIG. 1; and

FIG. 5 is a fragmentary cross-sectional view of a portion of the introduction instrument of FIG. 2.

Corresponding reference characters indicate corresponding parts throughout the several views. The exemplifications set out herein illustrate exemplary embodiments of the disclosure and such exemplifications are not to be construed as limiting the scope of the disclosure in any manner.

### DETAILED DESCRIPTION

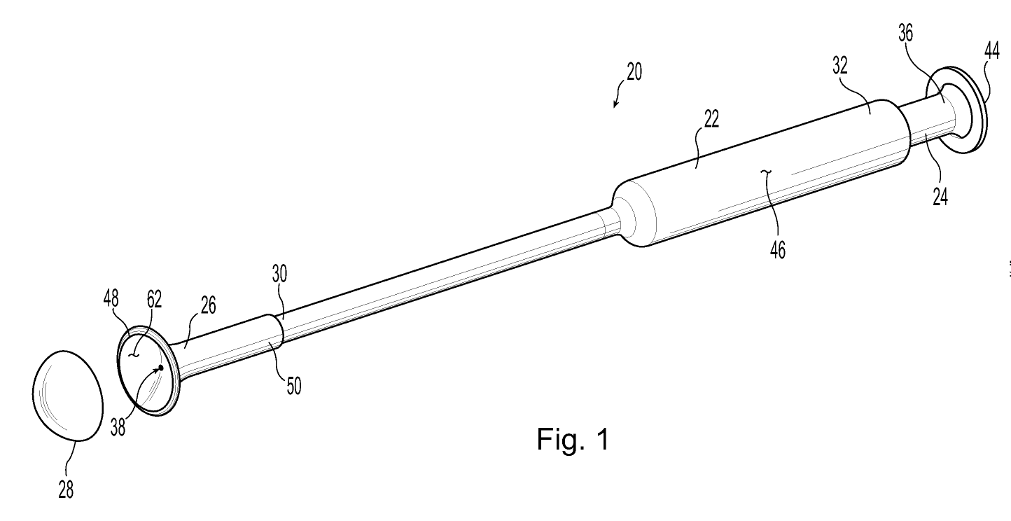

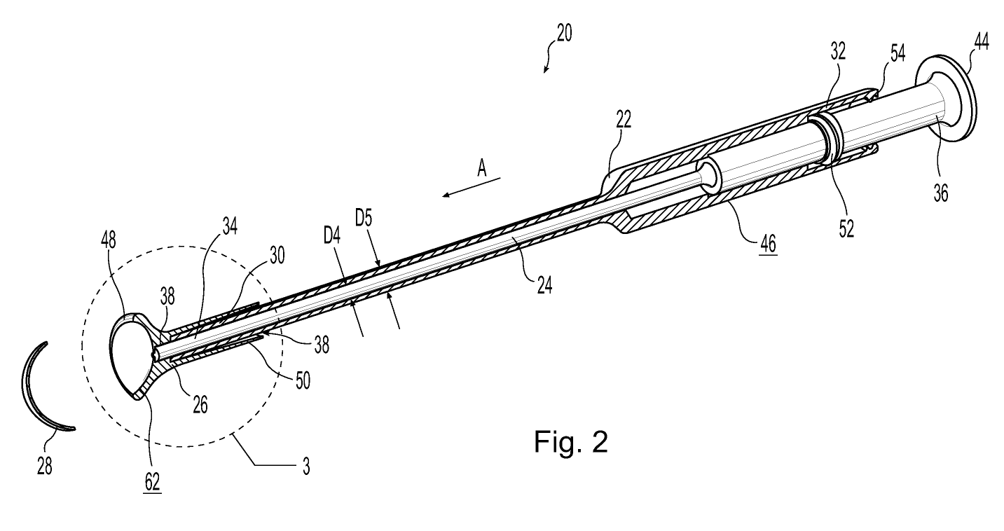

Referring to FIGS. 1 and 2, introduction instrument 20 is shown and generally includes outer housing or frame 22, inner shaft or plunger 24, and suction cup 26. Introduction instrument 20 may be used to insert liner component 28 into an acetabular shell (not shown) of an acetabular cup assembly. Outer housing 22 includes outer housing distal end 30 and outer housing proximal end 32. Outer housing 22 may include knurled surface 46 to enhance gripping friction of outer housing 22. Inner shaft 24 includes inner shaft distal end 34 and inner shaft proximal end 36. Inner shaft 24 may include proximal end face 44 for facilitating use of inner shaft 24 via a thumb, for example. Suction cup 26 includes suction cup cannula 38 extending throughout suction cup 26 from suction cup distal end 48 to suction cup proximal end 50. Suction cup 26 includes suction cup face 62.

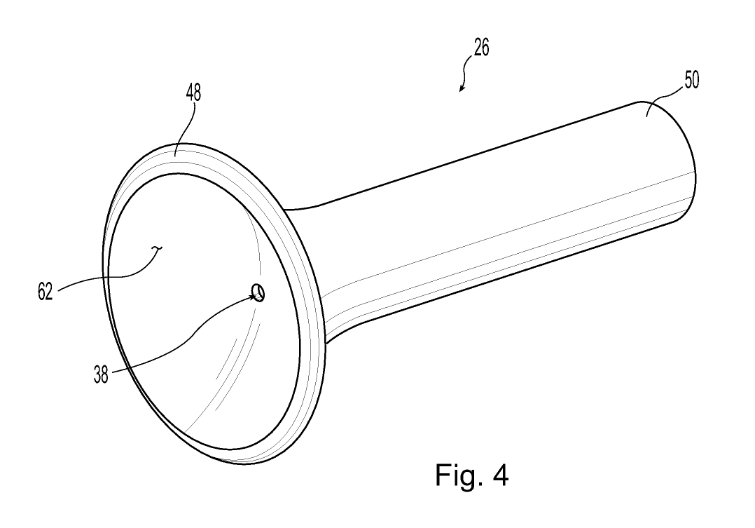

Referring now to FIGS. 2-5, suction cup 26 includes suction cup cannula first section 56, second section 58, and third section 60. The interface between first section 56 and second section 58 defines suction cup cannula first shoulder 40. The interface between second section 58 and third section 60 defines suction cup cannula second shoulder 42. Suction cup cannula first section 56 defines a first diameter D1, suction cup cannula second section 58 defines a second diameter D2, and suction cup cannula third section 60 defines a third diameter D3. In an exemplary embodiment, first diameter D1 is larger than second diameter D2 and second diameter D2 is larger than third diameter D3. In one embodiment, second diameter D2 is between approximately 0.1 and 0.2 inches. In an exemplary embodiment, second diameter D2 is approximately 0.125 inches. In one embodiment, first diameter D1 is between approximately 0.2 and 0.3 inches. In an exemplary embodiment, first diameter D1 is approximately 0.250 inches. In one embodiment, suction cup cannula first section 56 may taper towards suction cup distal end 48 such that first diameter D1 increases to between approximately 0.3 and 0.4 inches at proximal end 50 (while first diameter D1 near suction cup distal end 48 remains between approximately 0.2 and 0.3 inches), and, in an exemplary embodiment, first diameter D1 increases to approximately 0.330 inches at suction cup proximal end 50 (while first diameter D1 near suction cup distal end 48 remains approximately 0.250 inches). The taper connection between suction cup 26 and outer housing 22 facilitates connection and assembly therebetween. In one embodiment, third diameter D3 is between approximately 0.05 and 0.10 inches. In an exemplary embodiment, third diameter D3 is approximately 0.062 inches.

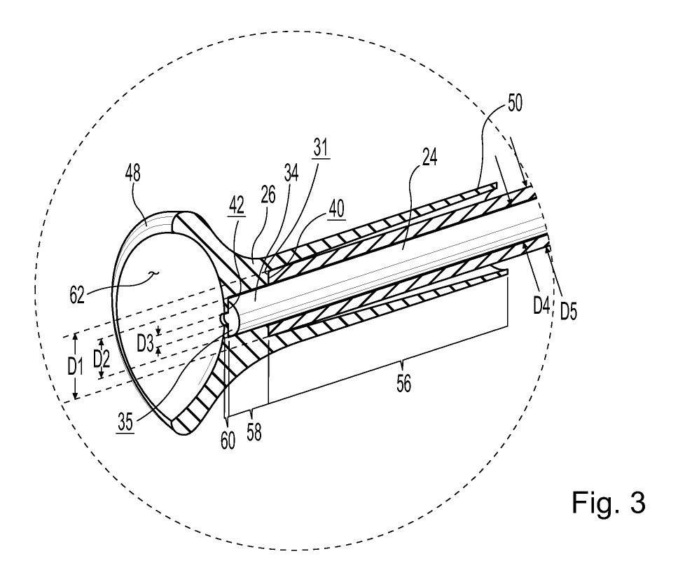

Referring still to FIGS. 2 and 3, inner shaft distal end 34 defines a fourth diameter D4 between approximately 0.1 and 0.2 inches. In an exemplary embodiment, fourth diameter D4 is approximately 0.170 inches. In an exemplary embodiment, inner shaft 24 is substantially cylindrical at distal end 34. Outer housing distal end 30 defines a fifth diameter D5 between approximately 0.250 and 0.3 inches. In an exemplary embodiment, fifth diameter D5 is approximately 0.280 inches. Outer housing 22 may be substantially cylindrical at distal end 30 or outer housing 22 may include a slight taper or barb feature towards distal end 30 to facilitate connection and assembly while resisting unintended disengagement between outer housing 22 and suction cup 26, as described below. In the tapered configuration, fifth diameter D5 increases to between approximately 0.35 and 0.4 inches proximate suction cup proximal end 50 when assembled, and, in an exemplary embodiment, fifth diameter D5 increases to approximately 0.360 inches proximate suction cup proximal end 50 when assembled.

To assemble introduction instrument 20 and referring now to FIG. 2, a user, e.g., a surgical technician, nurse, or surgeon, inserts inner shaft 24 into outer housing 22. Upon engagement of inner shaft exterior threads 52 with outer housing interior threads 54, the user rotates inner shaft 24 relative to outer housing 22 to threadingly engage each other. Once sufficient rotation is imparted to inner shaft 24 relative to outer housing 22, inner shaft exterior threads 52 move distally beyond outer housing interior threads 54 and become disengaged therefrom. At this point, outer housing interior threads 54 provide a barrier or stop to prevent unintended disengagement of inner shaft 24 from outer housing 22 and capture inner shaft 24 therein. Inner shaft 24 is allowed axial movement within outer housing 22 yet captured therein.

Suction cup 26 is assembled to outer housing 22 via friction-fit engagement between suction cup cannula first section 56 and outer housing distal end 30. As described above, outer housing distal end 30 includes fifth diameter D5 which is slightly larger than first diameter D1 of suction cup cannula first section 56, thereby slightly deforming suction cup cannula first section 56 upon introduction of outer housing 22 into suction cup 26 and enhancing the friction-fit engagement therebetween. In an exemplary embodiment, first diameter D5 is greater than first diameter D1 by approximately 0.03 inches to 0.04 inches. In one embodiment, outer housing distal end 30 includes a tapered section or barb feature to further enhance the engagement between outer housing 22 and suction cup 26. Suction cup 26 is pushed onto outer housing 22 until suction cup cannula first shoulder 40 abuts outer housing distal end surface 31. Inner shaft 24 is then distally pushed such that inner shaft distal end 34 engages suction cup cannula second section 58. As described above, inner shaft distal end 34 includes fourth diameter D4 which is slightly larger than second diameter D2 of suction cup cannula second section 58, thereby slightly deforming suction cup cannula second section 58 upon introduction of inner shaft 24 into suction cup 26 and enhancing the friction-fit engagement therebetween. In an exemplary embodiment, fourth diameter D4 is greater than second diameter D2 by approximately 0.04 inches to 0.05 inches. Inner shaft 24 is pushed into suction cup 26 until inner shaft distal end surface 35 abuts suction cup cannula second shoulder 42, thereby defining a first position of inner shaft 24 relative to outer housing 22 and suction cup 26. Instrument 20 is then ready to be assembled with liner 28.

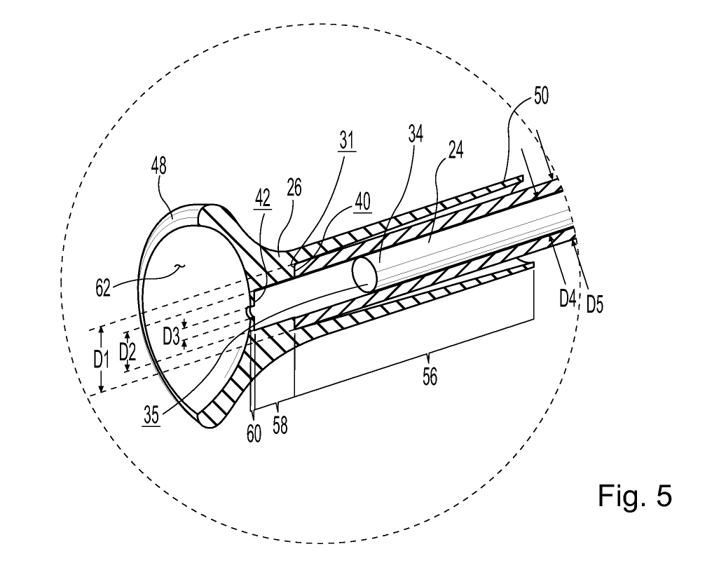

In operation, the user releasably attaches liner 28 to introduction instrument 20 by generally aligning suction cup 26 on an articulating surface of liner 28 and applying force in a direction generally along arrow A to force suction cup face 62 against liner 28 and thereby create a vacuum seal between introduction instrument 20 and liner 28. The user proceeds to implant liner 28 into a shell (not shown) of an acetabular cup assembly. Upon final seating of liner 28 in the shell, the user grasps outer housing 22 near knurled surface 46, for example, to statically hold outer housing 22 and proximally pulls inner shaft 24 relative to outer housing 22 to a position as shown in FIG. 5. Such movement of inner shaft 24 corresponding to a second position (FIG. 5) of inner shaft 24 relative to outer housing 22 and suction cup 26 releases the seal created between suction cup 26 and liner 28, thereby allowing introduction instrument 20 to release liner 28 therefrom without disturbing the seating relationship previously established with the shell.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. An instrument for introducing a liner into a shell of an acetabular cup, the instrument comprising:
an outer housing including an outer housing distal end and an outer housing proximal end;
an inner shaft captured within the outer housing, the inner shaft including an inner shaft distal end and an inner shaft proximal end; and
a suction cup including a cannula extending therethrough, the suction cup attached to the outer housing distal end, the inner shaft moveable from a first position relative to the outer housing and the suction cup to a second position relative to the outer housing and the suction cup, wherein the first position corresponds to an engagement of the suction cup and the liner and wherein the second position corresponds to a disengagement of the suction cup and the liner, wherein the inner shaft is moved from the first position to the second position by moving the inner shaft proximally.

2. The instrument of claim 1, wherein the cannula includes a first shoulder and a second shoulder, the first shoulder abutting the outer housing distal end and the second shoulder abutting the inner shaft distal end when the inner shaft is in the first position.

3. The instrument of claim 2, wherein the first shoulder defines a first cannula diameter and the second shoulder defines a second cannula diameter, the first cannula diameter greater than the second cannula diameter.

4. The instrument of claim 2, wherein the first shoulder defines a first cannula diameter and the outer housing distal end defines an outer housing diameter, the outer housing diameter being larger than the first cannula diameter such that a friction-fit engagement is effected between the outer housing and the suction cup.

5. The instrument of claim 2, wherein the second shoulder defines a second cannula diameter and the inner shaft distal end defines an inner shaft diameter, the inner shaft diameter being larger than the second cannula diameter such that a friction-fit engagement is effected between the inner shaft and the suction cup.

6. The instrument of claim 2, wherein the suction cup includes a proximal end and a distal end, the cannula tapering inward from the proximal end to the first shoulder, the distal end configured to engage the liner.

7. The instrument of claim 1, wherein the inner shaft distal end is engaged with the suction cup in the first position and disengaged with the suction cup in the second position.

8. The instrument of claim 1, wherein the inner shaft includes exterior threads proximate the inner shaft proximal end and the outer housing includes interior threads proximate the outer housing proximal end, the exterior threads and the interior threads cooperating to facilitate assembly of the outer housing and the inner shaft and to capture the inner shaft within the outer housing upon assembly completion.

9. A system for orthopaedic surgery, the system comprising:
an acetabular cup liner; and
a liner insertion instrument, the insertion instrument comprising:
an outer housing including an outer housing distal end, an outer housing proximal end, an outer housing cannula extending through the outer housing from the outer housing distal end to the outer housing proximal end, and interior threads in the outer housing cannula proximate the outer housing proximal end;
an inner shaft including an inner shaft distal end, an inner shaft proximal end, and exterior threads on the inner shaft proximate the inner shaft proximal end, the exterior threads engageable with the interior threads in the outer housing cannula to capture the inner shaft within the outer housing; and
a suction cup configured for engagement with the liner, the suction cup including a suction cup cannula extending therethrough, the suction cup attached to the outer housing distal end, wherein the suction cup cannula includes a first shoulder and a second shoulder, the first shoulder abutting the outer housing distal end, wherein the first shoulder defines a first cannula diameter and the second shoulder defines a second cannula diameter, the first cannula diameter greater than the second cannula diameter, and wherein the suction cup includes a proximal end and a distal end.

10. The system of claim 9, wherein after the exterior threads of the inner shaft are threaded distally past the interior threads of the outer housing cannula, the inner shaft is axially moveable within the outer housing cannula and constrained from removal from the outer housing cannula.

11. The system of claim 9, wherein the inner shaft is moveable from a first position relative to the outer housing and the suction cup to a second position relative to the outer housing and the suction cup, wherein the first position corresponds to an engagement of the suction cup and the liner and wherein the second position corresponds to a disengagement of the suction cup and the liner, wherein the inner shaft is moved from the first position to the second position by moving the inner shaft proximally.

12. The system of claim 11, wherein the suction cup cannula first shoulder abuts the outer housing distal end and the suction cup cannula second shoulder abuts the inner shaft distal end when the inner shaft is in the first position.

13. The system of claim 12, wherein the inner shaft distal end is engaged with the suction cup in the first position and disengaged with the suction cup in the second position.

14. The system of claim 9, wherein the outer housing distal end defines an outer housing diameter, the outer housing diameter being larger than the first cannula diameter such that a friction-fit engagement is effected between the outer housing and the suction cup.

15. The system of claim 9, wherein the inner shaft distal end defines an inner shaft diameter, the inner shaft diameter being larger than the second cannula diameter such that a friction-fit engagement is effected between the inner shaft and the suction cup.

16. A method for inserting an acetabular cup liner of an acetabular cup assembly including the acetabular cup liner and an acetabular cup shell, the method including the steps of:
assembling a liner introduction instrument, the steps of assembling comprising:
inserting an inner shaft into an outer housing;
capturing the inner shaft within the outer housing by rotating the inner shaft distally beyond an engagement of interior threads of the outer housing and exterior threads of the inner shaft;
attaching a suction cup to a distal end of the outer housing; and
engaging the inner shaft with the suction cup;
engaging the liner to the liner introduction instrument by pressing the suction cup into the liner;
inserting the liner into the shell; and
disengaging the liner from the liner introduction instrument by moving the inner shaft proximally while the outer housing remains statically positioned, thereby releasing a seal of the suction cup.

17. The method of claim 16, wherein the step of attaching the suction cup to the distal end of the outer housing comprises press-fitting a cannula of the suction cup onto the distal end of the outer housing.

18. The method of claim 17, wherein the step of press-fitting comprises pushing the suction cup onto the distal end of the outer housing until a first shoulder of the cannula engages the distal end of the outer housing.

19. The method of claim 16, wherein the step of engaging the inner shaft with the suction cup comprises press-fitting a distal end of the inner shaft into the cannula of the suction cup.

20. The method of claim 19, wherein the step of press-fitting comprises pushing the inner shaft into the suction cup cannula until a second shoulder of the cannula engages the distal end of the inner shaft.
